(19) United States
(12) Patent Application Publication
(10) Pub. No.: US 2006/0275231 A1
(43) Pub. Date: Dec. 7, 2006

(54) Cosmetic composition, a packaging device and a method of application

(75) Inventor: Christophe Dumousseaux, Tokyo (JP)

Correspondence Address: OLIFF & BERRIDGE, PLC, P.O. BOX 19928, ALEXANDRIA, VA, 22320, US

(73) Assignee: L'OREAL, Paris (FR)

(21) Appl. No.: 11/430,229

(22) Filed: May 9, 2006

Related U.S. Application Data
(60) Provisional application No. 60/714,840, filed on Sep. 8, 2005.

(30) Foreign Application Priority Data
May 10, 2005 (FR) 05 51214

(51) Int. Cl. A61K 8/18

(52) U.S. Cl. 424/63; 424/64

(57) ABSTRACT

A cosmetic composition, in a physiologically acceptable medium, may include at least one photoluminescent agent. The at least one photoluminescent agent may cause at least three photoluminescent peaks to be observed at respective different wavelengths in the visible spectrum when the composition is illuminated at a wavelength of 365 nm.

This non-provisional application claims the benefit of French Application No. 05 51214 filed on May 10, 2005, and U.S. Provisional Application No. 60/714,840 filed on Sep. 8, 2005, the entire disclosures of which are incorporated herein by reference.

The present invention relates to a cosmetic composition, for example, a cosmetic composition for skin care and/or makeup, for example, of the body, the hands, the neck or the face, the lips, and/or keratinous fibers, for example, eyebrows, eyelashes, or hair.

The composition of the invention may seek more particularly to lighten the skin of the face and/or to camouflage skin defects.

### BACKGROUND

To lighten the skin, it is known to use photoluminescent organic molecules, for example, brighteners, for example, those described in patent application EP 0 962 224.

It is also known to lighten the skin by using compositions including photoluminescent inorganic compounds based on precious stones, such as those described in U.S. Pat. No. 6,753,002, or based on doped metallic oxides, such as those described in Japanese Patent No. 2,805,373.

### SUMMARY

The photoluminescent intensity of certain brighteners is satisfactory, but the use of such molecules is not desirable in the field of cosmetics because of their potential toxicity.

In addition, photoluminescent organic molecules present poor stability in light and thus lose their effectiveness over time after application of the composition to the skin.

The photoluminescent intensity of photoluminescent inorganic compounds is very low, which means that it is not possible to obtain a satisfactory lightening effect on application to the skin.

There thus exists a need to have a cosmetic composition available that provides satisfactory lightening properties.

There also exists a need to benefit from cosmetic compositions that provides novel optical properties, that are non-toxic, and that behave in a stable manner over time, for example, in a photostable manner.

There also exists a need to act on skin tone, to provide protection against solar ultraviolet light, or to create novel makeup colors.

Exemplary embodiments of the invention may make it possible to satisfy all or some of those needs, amongst others.

Exemplary embodiments of the invention may provide a novel cosmetic composition comprising, in a physiologically acceptable medium, at least one photoluminescent agent, preferably an inorganic agent, selected in such a manner that at least three photoluminescent peaks are observed at three respective different wavelengths in the visible spectrum, when the composition is illuminated at a wavelength of 365 nanometers (nm).

In exemplary embodiments, the composition may comprise at least three different photoluminescent agents, which may be associated respectively with the peaks, and which may preferably be inorganic.

Exemplary embodiments of the invention may provide a cosmetic composition comprising, in a physiologically acceptable medium, at least three different photoluminescent agents.

In such exemplary embodiments, the three photoluminescent agents may be selected in such a manner that at least three respective photoluminescent peaks are observed at different wavelengths when the composition is illuminated under ultraviolet light.

When evaluating photoluminescence, the ultraviolet light used for characterizing the photoluminescence may have a wavelength shorter than 400 nm, and may essentially have a wavelength of 365 nm, for example, by using filters and/or diffraction gratings.

In exemplary embodiments, the plurality of photoluminescent peaks makes it possible to create new colors by additive synthesis, and, for example, to create substantially white light or any other light that improves the tone (natural skin color) or reinforces the color of an additional colorant (dyestuff) included in the composition.

In exemplary embodiments, the colors emitted by photoluminescence may, for example, be complementary colors, such as red, green, and blue, for example.

Exemplary embodiments of the invention may provide a pigment comprising at least two, better three, photoluminescent agents at least partially coated by a preferably transparent matrix, a mean size of the pigment particles preferably being less than 100 nm.

For example, the matrix may include a refractive index that is less than or equal to 1.8, better less than or equal to 1.6.

Exemplary embodiments of the invention may provide a cosmetic composition including such a pigment.

In exemplary embodiments, a spectral distance between two of the three peaks may be greater than or equal to about 40 nm, or indeed about 60 nm. The spectral distance between any two of the three peaks may be greater than or equal to about 60 nm.

In exemplary embodiments, an intensity ratio between a peak of greatest intensity and a peak of lowest intensity may be less than or equal to 20, or even 10, or 5, or 3. The intensity ratio between any two of the three peaks may be less than or equal to 20, or even 10, or 5, or 3. All of the peaks may also have substantially a same intensity. A small intensity ratio between the peaks may make it easier to create white light by additive synthesis.

Exemplary embodiments of the invention may provide a cosmetic composition comprising, in a physiologically acceptable medium, at least one photoluminescent agent selected in such a manner that at least three photoluminescent peaks are observed having respective different wavelengths in the visible spectrum when the composition is illuminated under ultraviolet light, a ratio between a peak of greatest intensity and a peak of smallest intensity being less than or equal to 20, or even 10, or 5, or 3.

In exemplary embodiments, various proportions of the photoluminescent agents may be selected in such a manner as to obtain desired light intensities. Certain agents may thus be included in greater proportions if they include lower emissivities.

In exemplary embodiments, a peak may have a wavelength lying, for example, in a range of about 620 nm to about 700 nm, or about 592 nm to about 620 nm, or about 578 nm to about 592 nm, or about 500 nm to about 578 nm, or about 446 nm to about 500 nm, or about 400 nm to about 446 nm. Various wavelengths may be selected in such a manner as to create light of a predefined color by additive synthesis, for example, white light or light providing a predefined tone, for example, corresponding to a skin tone/complexion.

Where appropriate or desired, the wavelengths selected and/or the relative proportions of the various photoluminescent agents may be personalized as a function of the calorimetric characteristics of the medium being made up. The composition may then be formulated on a site of sale, for example, with formulation being preceded by taking a measurement of optical properties of a user's skin. In a composition corresponding to an exemplary embodiment, a first peak has a wavelength lying in a range of about 446 nm to about 500 nm, a second peak has a wavelength lying in a range of about 500 nm to about 578 nm, and a third peak has a wavelength lying in a range of about 620 nm to about 700 nm. The photoluminescent agents may thus emit respectively in the blue, in the green, and in the red.

In another exemplary composition, a first peak has a wavelength lying in a range of about 400 nm to about 446 nm, a second peak has a wavelength lying in a range of about 500 nm to about 578 nm, and a third peak has a wavelength lying in a range of about 592 nm to about 620 nm. The photoluminescent agents may then emit respectively in the violet, green and orange color spectrums.

In yet another composition, a first peak has a wavelength lying in a range of about 446 nm to about 500 nm, a second peak has a wavelength lying in a range of about 578 nm to about 592 nm, and a third peak has a wavelength lying in a range of about 620 nm to about 700 nm. Under such circumstances, the photoluminescent agents emit respectively in the blue, yellow and red color spectrums.

Measuring Photoluminescence

For example, photoluminescence may be measured by using the FP6200 spectrofluorimeter from the supplier Jasco Corporation, fitted to perform excitation or emission scanning over a range 220 nm to 730 nm. For example, the width of the excitation band and of the emission band is set at 5 nm and acquisition is undertaken using a step size of 1 nm.

The composition is applied in a pasty or liquid state as a film having a thickness lying in a range of about 20 micrometers (μm) to about 100 μm on the black portion of a contrast card from Nippon Test Panel.

The composition is then dried for two hours at ambient temperature.

The contrast card is then placed on an FDA-430 sample carrier sold by the supplier Jasco Corporation, and the emission spectrum is measured in reflection for an excitation wavelength of 365 nm, with the width of the excitation band and of the emission band being equal to 5 nm.

When the composition is in the form of a powder, the composition is initially placed in an FP-1061 cell sold by the supplier Jasco Corporation.

The cell is then mounted on the FDA-430 sample carrier likewise sold by the supplier Jasco Corporation. The emission spectrum is then measured in reflection for an excitation wavelength of 365 nm.

Photoluminescent Agents

In exemplary embodiments, the photoluminescent agents may include a variety of chemical and physical structures.

At least one photoluminescent agent may be inorganic or organic.

At least one photoluminescent agent may be a pigment or a colorant.

At least one photoluminescent agent may comprise a doped metallic compound, for example, a doped metallic salt, where the metallic salt may, for example, comprise zinc sulfide, and the dopant may comprise at least one metal. For example, the metallic salt may comprise particles having a mean size of less than 15 μm, for example, 10 μm, for example, less than 5 μm.

At least one photoluminescent agent may comprise a doped phosphate or aluminate, where the dopant may comprise at least one rare earth, for example.

At least one photoluminescent agent may comprise at least one oxide of at least one rare earth.

The mean size of the particles of at least one photoluminescent agent may be less than or equal to about 100 nm, or indeed about 50 nm. A relatively small mean size for the particles may make it possible to increase the intensity of photoluminescence, and thus, for example, to increase a desired lightening effect.

The term “mean size” as applied to particles is used herein to designate the size given at the half population point in the statistical distribution of grain sizes, and is written D₅₀. This size may be measured using an electron microscope, for example.

At least one photoluminescent agent may comprise particles coated at least in part by a matrix, which may be transparent. The particles may be totally encapsulated in the matrix. The matrix may be advantageous, for example, when the coated particles are of nanometer size, for example, when the coated particles have a mean size that is less than or equal to 100 nm, better 50 nm, or even better 20 nm. The matrix may be polymeric or inorgamc.

The matrix may include a refractive index that is less than or equal to about 1.8, or indeed less than or equal to about 1.6.

When the matrix is polymeric, the matrix may comprise, for example, polystyrene (PS), polymethylmethacrylate (PMMA), polyester, polyethylene (PE), or polyether, for example, in particulate form.

Particles of PMMA, PE, PS, or polycarbonate (PC) powder encapsulating nanoparticles of CdSe are sold by the supplier Evident Technologies under the name Evi Composite™.

When the matrix is inorganic, the matrix may comprise particles of silica, for example.

The matrix may comprise particles containing at least one or even two or even three different photoluminescent agents.

The matrix may be advantageous for isolating the photoluminescent agent(s) from the surrounding medium.

The total proportion of photoluminescent agents in the composition may lie, for example, in a range of about 1% to about 50% by weight, relative to a total weight of the composition, for example, in a range of about 5% to about 20% by weight, compared with a total weight of the composition. This proportion may be selected as a function of a desired saturation and/or covering power, for example.

In exemplary embodiments, at least one photoluminescent agent may comprise particles of a mean size lying in a range of about 1 nm to about 10 nm, for example, in the form of crystals of CdSe, of CdSe/ZnS, or of CdTe/CdS.

In other exemplary embodiments, at least one photoluminescent agent may comprise a semiconductor, for example, in particulate form, for example, Si, ZnO, CdSe, and the like.

The number of photoluminescent agents may be greater than three.

Amongst suitable commercially available photoluminescent agents, mention may be made, amongst others, of the Lumilux® photoluminescent agents sold by the supplier Honeywell, for example, Lumilux® Effect Blue A, Lumilux® Effect Green A, and Lumilux® Effect Red A, or the Netoje® photoluminescent agents containing a high proportion of rare earths and sold by the supplier Nec/Tokin.

Additional Colorant

In addition to the photoluminescent agent, the composition may include at least one additional colorant. The term “colorant” is used herein to designate any organic or inorganic additive capable of producing a color effect.

The additional colorant maybe included at a concentration of 0.01% to 20% by weight, for example, 0.05% to 10% by weight, for example, 0.1% to 7% by weight, for example, 0.1% to 5% by weight relative to a total weight of the composition.

Examples of colorants include liposoluble colorants, hydrosoluble colorants, pigments, and pearlescent nacres commonly used in dermatological or cosmetic compositions, and mixtures thereof.

For example, liposoluble colorants include Sudan red, DC red 17, DC green 6, β-carotene, soy oil, Sudan brown, DC yellow 11, DC violet 2, DC orange 5, and quinoline yellow.

The pigments may be white or colored, inorganic and/or organic, covered or uncovered.

The pigments may be selected, for example, from inorganic pigments, titanium oxide, possibly surface-treated, the oxides of zirconium or cerium, and also the oxides of iron or chromium, manganese violet, ultramarine blue, chromium hydrate, and ferric blue.

Amongst organic pigments, mention may be made of carbon black, the pigments listed in the D&C classification, and lakes based on cochineal carmine, on barium, strontium, calcium and/or aluminum.

Nacreous pigments may be selected from white nacreous pigments, such as titanium-covered mica or bismuth oxychloride, colored nacreous pigments, such as titanium mica with iron oxides, titanium mica with ferric blue or chromium oxide, for example, titanium mica with an organic pigment of the above-specified type, and nacreous pigments based on bismuth oxychloride.

The pigments may be subjected to surface treatment.

Effect pigments may also be used, for example, interference pigments.

Packaging and/or Applicator Devices

The composition may be packaged in multiple forms, with or without an applicator, depending on a dosage form of the composition.

The composition may be packaged in a packaging device such as a box, receptacle, or case, that is leaktight, at least before first use. The packaging device may be made at least in part using a thermoplastic material, or alternatively without using any thermoplastic material. The packaging device may comprise a polyolefin. The packaging device may also include at least one metal element, for example, a cup, a twisted metal core, a hinge, a ring, or a cover.

When the composition is for application by an applicator, the applicator may comprise, for example, a foam, an optionally flocked endpiece, a felt, a brush, a comb, a paintbrush, or a piece of woven or non-woven fabric.

The composition may also impregnate a substrate, for example, a paper, or a woven or non-woven fabric.

In embodiments that include an applicator, the applicator may be received removably on the packaging device containing the composition. Alternatively, the applicator may be permanently secured to the packaging device containing the composition. The packaging device may include a piston or any other means for enabling the applicator to be fed with the composition.

The packaging device may include a dispenser member such as a pump or a valve, for example, in embodiments in which the composition is liquid.

In embodiments that include an applicator, the applicator may comprise a stem connected to a member configured to close the packaging device, which closure member may also constitute a handle member, where appropriate or desired.

The closure device containing the composition may be provided with a catch or any other fastener means, for example, magnetic means or snap-fastener means.

The packaging device may also be provided with fastener means engaged by screw-fastening, friction or snap-fastening.

The packaging device may include sealing means such as, for example, an annular sealing lip or an elastomer gasket, either injection-molded onto the device or fitted thereto.

The packaging device containing the composition may carry a label or printing, for example, showing a trademark or a logo, with the printing being performed, for example, by hot- or cold-transfer or by silkscreen printing or by some other printing technique.

The packaging device containing the composition may comprise a card package or a blister pack, for example, being made in part out of transparent plastics material.

Makeup Method

Exemplary embodiments of the invention may provide a method of applying makeup and/or a non-therapeutic care product to skin, lips, and/or keratinous fibers. The method may include a step of applying a composition as defined above to skin, lips, and/or keratinous fibers. Before performing such step, it is possible to perform a measurement of optical properties to formulate the composition accordingly.

Physiologically Acceptable Medium

Exemplary embodiments of the composition of the invention comprise at least one physiologically acceptable medium.

The term “physiologically acceptable medium” is used herein to designate a medium that is not toxic and that is suitable for application to skin, lips, or keratinous material of human beings. The physiologically acceptable medium is generally adapted to the nature of the substrate onto which the composition is to be applied and also to the way in which the composition is to be packaged.

### BRIEF DESCRIPTION OF THE DRAWINGS

FIG. 1 is a diagram illustrating an exemplary photoluminescence spectrum of a composition as a function of wavelength.

### DETAILED DESCRIPTION OF EMBODIMENTS

In certain embodiments, the composition of the invention may include a fatty phase, for example, comprising 5% to 80% by weight, and for example, 5% to 50% by weight relative to the total weight of the composition.

Oils that may be used in the composition may be selected from those conventionally used in the field.

The composition may include an oil, for example, an oil selected from:

hydrocarbon oils of animal origin, such as perhydrosqualene;

hydrocarbon oils of vegetable origin, such as liquid triglycerides of fatty acids having 4 to 10 carbon atoms, and the liquid fraction of shea butter;

synthesized ethers and esters, for example, of fatty acids, such as oils having formulae R¹COOR² and R¹OR² where R¹ represents the residue of a fatty acid or a fatty alcohol having 8 to 28 carbon atoms, and R² represents an optionally branching hydrocarbon chain containing 3 to 30 carbon atoms, such as, for example, Purcellin oil, isononyl isononanoate, isopropyl myristate, ethyl-2-hexyl palmitate, octyl-2-dodecyl stearate, octyl-2-docecyl erucate, isostearyl isostearate; hydroxyl esters such as isostearyl lactate, octylhydroxystearate, octyldocecyl hydroxystearate, diisotearyl malate, triisocetyl citrate, hepatanoates, octanoates, decanoates of fatty alcohols; polyol esters such as propylene glycol dioctanoate, neopentylglycol diheptaonoate, and diethyleneglycol diisononanoate; and esters of pentaerythritol such as pentaerythrityl tetraisostearate;

linear or branching hydrocarbons of mineral or synthetic origin, such as optionally volatile paraffin oils and derivatives thereof, Vaseline, polydecenes, hydrogenated polyisobutene such as parleam oil;

fatty alcohols having 8 to 26 carbon atoms, such as cetylic alcohol, stearylic alcohol, and mixtures thereof (cetylstearylic alcohol), octyldodecanol, 2-butyloctanol, 2-hexyldecanol, 2-undecylpentadecanol, oleic alcohol, or linoleic alcohol;

partially hydrocarbon and/or silicone fluorinated oils such as those described in document JP-A-2-295 912;

silicone oils such as optionally volatile polymethylsiloxanes (PDMS) having a linear or cyclic silicone chain, that are liquid or pasty at ambient temperature, for example, cyclopolydimethylsiloxanes (cylmethicones) such as cyclohexasiloxane; polydimethylsiloxanes having alkyl, alcoxy, or phenyl groups, either pendant or at the end of the silicone chain, the groups having 2 to 24 carbon atoms; phenyl silicones such as phenyltrimethicones, phenyldimethicones, phenyltrimethylsiloxydiphenyl-siloxanes, diphenyl-dimethicones, diphenylmethyldiphenyl trisiloxanes, 2-phenylethyltrimethyl-siloxysilicates, and polymethylphenylsiloxanes; and

mixtures thereof.

When the composition is in the form of an emulsion having a fatty phase, the composition may also include an emulsifier, and optionally a coemulsifier.

As examples of emulsifiers and coemulsifiers suitable for use in exemplary embodiments the invention, mention may be made of O/W emulsifiers such as fatty acid esters and polyethylene glycol, for example, PEG-100 stearate, and fatty acid esters and glycerine such as glyceryl stearate, and also W/O emulsifiers such as oxyethylene poly(methylcetyl)(dimethyl)methylsiloxane available under the commercial name Abi WE09 from the supplier Degussa Goldschmidt or the mixture of ethylene glycol acetyl stearate and glyceryl tristearate sold by the supplier Guardian under the trade name Unitwix.

The emulsifier and optionally the coemulsifier may generally be included in the composition at a proportion lying in a range 0.3% to 30% by weight, and, for example, lying in a range 0.5% to 20% by weight relative to a total weight of the composition.

Exemplary embodiments of the composition of the invention may also include at least one wax, at least one gum, and at least one pasty fatty substance of vegetable, animal, mineral, or synthetic origin, optionally treated with silicone.

The waxes may be hydrocarbon, silicone, and/or fluorinated waxes, optionally made up of ester or hydroxyl functions. The waxes may be of natural origin, for example.

The wax may represent 0.01% to 10% by weight, and for example, 0.1% to 5% by weight relative to a total weight of the composition. In exemplary embodiments, the composition may be free of any wax.

The composition may also include at least one optionally coated organic or inorganic filler, for example, as an anti-shine agent, for example, zinc and zirconium oxide, silica, alumina, boron nitride, talc, sericite, mica, clays, starch and derivatives thereof, for example, the starch cross-linked with octenylsuccincic anhydride and sold by the supplier National Starch under the name DRY FLO PLUS (28-1160), aqueous dispersions of acrylic styrene, particles of melamine-formaldehyde or urea-formaldehyde resin, aqueous dispersions of polytetrafluoroethylene, microdispersions of waxes, copolymers of vinylpyrrolidone and 1-triacontene, hydrodispersible polymers containing LCST units, silicone resins and waxes, for example, microbeads of silicone resin such as those sold under the name TOSPEARL by the supplier Toshiba Silicone, expanded powders such as hollow microspheres and for example, the microspheres sold under the name EXPANCEL by the supplier Kemanord Plast or under the name MICROPEARL F 80 ED by the supplier Matsumoto, microspheres of expanded vinylidene chloride, acrylonitrile, and methacrylate terpolymer, polyamide particles, for example, Nylon® particles or those sold under the name ORGASOL by the supplier Atochem, microbeads of cellulose, fibers, powers of polyethylene, microspheres based on acrylic copolymers such as microspheres of ethylene glycol dimethacrylate and lauryl methacrylate copolymer sold by the supplier Dow Coming under the name POLYTRAP, and mixtures thereof.

The filler, for example, when used as an anti-shine agent, may be included at a concentration lying in a range 0.1% to 80% by weight relative to a total weight of the composition, for example, a concentration lying in a range 0.1% to 10% by weight relative to the total weight of the composition.

Naturally, the anti-shine agent and the quantity of agent used may be selected so as to avoid deteriorating the desired properties.

The composition may also contain at least one additive that is conventional in the field of cosmetics, such as fillers, for example, selected from the above list, hydrophilic or lipophilic gelling agents, hydrosoluble or liposoluble agents, preservatives, hydrating agents such as polyols and, for example, glycerin, sequestering agents, antioxidants, solvents, fragrances, physical and chemical sun filters, for example, against UVA and/or UVB, odor absorbers, (acidic or basic) pH adjusters, and mixtures thereof.

The quantities of these various additives may be those conventionally used in the field, for example, 0.01% to 20% of a total weight of the composition.

In any event, the additives, and the proportions thereof, may be selected in such a manner as to avoid degrading the desired properties.

As active agents, mention may be made, for example, of the following:

agents known for their activity on aging of the skin such as keratolytic and peeling-enhancing agents, for example, α-hydroxy acids, β-hydroxy acids, α-aceto acids, β-aceto acids, retinoids and esters thereof, retinal, retinoic acid, and derivatives thereof;

vitamins, such as for example vitamins A, B3, PP, B5, E, K1 and/or C, and derivatives of the vitamins, and, for example, their esters;

free radical scavengers;

sun filters;

hydrating agents such as polyols;

ceramides;

DHEA and derivatives thereof;

the coenzyme Q10;

dipigmenting and lightening agents acting in complementary biological manner such as kojic acid, extracts of scutellaria, mulberry, licorice, and/or camomile, derivatives of para-aminophenols, arbutine and derivatives thereof, and mixtures thereof;

agents suitable for use on fatty or mixed skin, such as salts of zinc, and, for example, zinc oxide and zinc gluconate;

antibacterial agents such as salicylic acid and derivatives thereof such as n-octanoyl-5-salicylic acid, triclosan, lipacid, capryloylglycine, clove extract, octopirox, hexamidine, azelaic acid, and derivatives thereof;

anti-acne agents, or indeed extracts from phlebotonic plants such as extracts of ruscus and/or horse chestnut; xanthic bases such a caffeine.

In exemplary embodiments, the composition may be in any of the dosage forms used in the field of cosmetics, and normally used for topical application: direct, inverse, or multiple emulsions; gels; creams; solutions; suspensions; lotions; free powders; compacts; and sticks.

More precisely, the composition may be in the form of an optionally gelled oily solution of an emulsion having a liquid or semiliquid consistency of the lotion type obtained by dispersing a fatty phase in an aqueous phase (O/W) or conversely an aqueous phase in a fatty phase (W/O), a triple emulsion (W/O/W/ or O/W/O), or a suspension or an emulsion of soft, semi-solid, or solid consistency of the cream or gel type, or indeed microemulsions, microcapsules, microparticles, or a vesicular dispersion of ionic type (liposomes or oleosomes) and/or of non-ionic type (niosomes) and/or a dispersion of nanocapsules or nanospheres.

The cosmetic composition may be, for example, in the form of a skin-care, makeup, and/or sunscreen composition.

More particularly, the composition may be in the form of makeup for the face, for example, the skin and/or the lips, for example, in the form of a foundation.

The composition may also be in the form of a skin-care and/or makeup composition for the skin, the lips, and/or keratinous fibers.

For example, the composition may be in the form of a concealer gel, a skin-care cream, or a photoprotective lotion, for example, against UV.

### EXAMPLES

The examples given below are by way of illustration and are not limiting. The percentages are expressed by weight relive to a total weight of the composition.

### Example 1

Foundation

| | % |
|---|---|
| Talc | 37.8 |
| Sericite | 31.5 |
| Mica | 4.5 |
| Titanium dioxide | 4.9 |
| Iron oxides | 2.0 |
| Zinc stearate | 0.9 |
| Liquid paraffin | 3.6 |
| Phenyl trimethicone | 4.5 |
| Parabens | 0.3 |
| Lumilux Effect Blue A* | 3 |
| Lumilux Effect Green A** | 2 |
| Lumilux Effect Red A*** | 5 |

The Lumilux ® photoluminescent agent is sold by the supplier Honeywell.
*The photoluminescent agent Lumilux Effect Blue A comprises silver-doped zinc sulfide (ZnS:Ag) in the form of particles having a mean size of 20 μm. The photoluminescent agent Lumilux Effect Blue A emits in the blue color spectrum with a peak of 450 nm and a broad emission band in a range of 400 nm to 520 nm under UV light.
**The photoluminescent agent Lumilux Effect Green A comprises copper-doped zinc sulfide (ZnS:Cu) in the form of particles having a mean size of 8 μm. The photoluminescent agent Lumilux Effect Green A emits in the green color spectrum with a peak at 530 nm and a broad emission band in a range of 420 nm to 660 nm under UV light.
***The photoluminescent agent Lumilux Effect Red A comprises europium-doped yttrium sulfide (Y₂O₂S:Eu) in the form of particles presenting a mean size of 10 μm. The photoluminescent agent Lumilux Effect Red A emits in the red color spectrum with a maximum at 620 nm and a relatively narrow emission band under UV light.

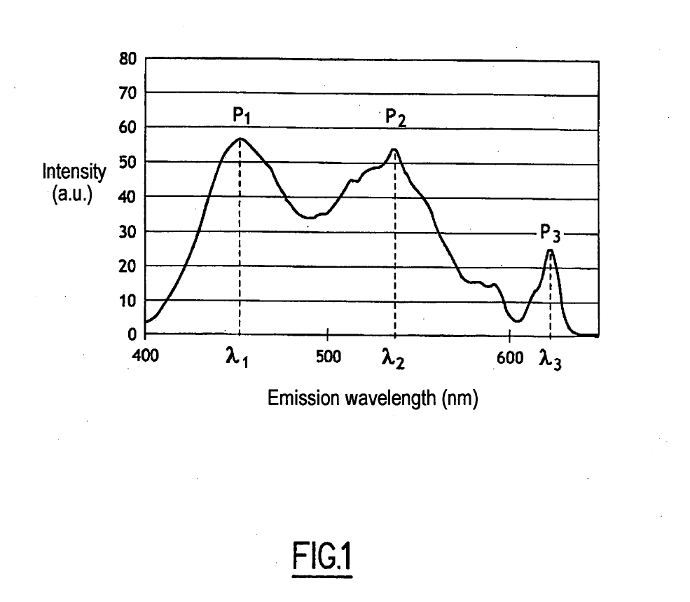

FIG. 1 is a diagram showing the photoluminescence spectrum of the composition of Example 1 as a function of wavelength.

In this example, the peak P1 has a wavelength λ₁ of about 450 nm, the corresponding photoluminescent agent having an emission band lying in a range of 400 nm to 520 nm, approximately. The peak P2 has a wavelength λ₂ of about 530 nm, the corresponding photoluminescent agent having an emission band lying in a range of 420 nm to 660 nm. Finally, the peak P3 has a wavelength λ₃ of 620 nm, the photoluminescent agent having an emission band lying in a range of 600 nm to 635 nm.

### Example 2

Liquid Foundation

| | % |
|---|---|
| Phase I | |
| Cetyl dimeticone copolyol/ | 8 |
| polyglycerol-4 isostearate/ | |
| hexyl laurate | |
| Dimethicone | 4.8 |
| Cyclomethicone | 5.2 |
| Isododecane | 2.8 |
| Isotearyl neopentanoate | 0.8 |
| Bentone gel | 8 |
| Titanium oxide | 5 |
| Iron oxide | 1 |
| Phase II | |
| Water | 43.2 |
| Butylene glycol | 5.6 |
| Magnesium sulfate | 0.8 |
| Preservatives | 0.8 |
| Phase III | |
| Talc | 4 |
| Netoje A-B¹ | 3 |
| Netoje S-G² | 2 |
| Netoje A-R³ | 5 |

Netoje ® particles are sold by the supplier Nec/Tokin.
¹The photoluminescent agent Netoje A-B emits in the blue color spectrum under excitation at 365 nm.
²The photoluminescent agent Netoje S-G emits in the green color spectrum under excitation at 365 nm.
³The photoluminescent agent Netoje A-R emits in the red color spectrum under excitation at 365 nm.

Preparation

Phase I and II are mixed separately, phase II is then added to phase I using a rotor agitator suitable for achieving emulsification. Thereafter, phase III is added to the emulsion.

Although various details of the present invention herein have been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention.

The expression “comprising a” should be understood as being synonymous with the expression “comprising at least one” unless specified to the contrary. Ranges should be understood as including the limits stated, unless specified to the contrary.

What is claimed is:

1. A cosmetic composition comprising, in a physiologically acceptable medium, at least one photoluminescent agent, wherein the at least one photoluminescent agent causes at least three photoluminescent peaks to be observed at respective different wavelengths in the visible spectrum when the composition is illuminated at a wavelength of 365 nm.

2. A cosmetic composition comprising, in a physiologically acceptable medium, at least three different photoluminescent agents.

3. A composition according to claim 1, wherein the at least one photoluminescent agent comprises at least three different photoluminescent agents.

4. A cosmetic composition according to claim 2, wherein the three photoluminescent agents cause at least three photoluminescent peaks to be observed associated with the respective photoluminescent agents, the peaks having different respective wavelengths when the composition is illuminated under ultraviolet light.

5. A cosmetic composition comprising, in a physiologically acceptable medium, at least one photoluminescent agent, wherein the at least one photoluminescent agent causes at least three photoluminescent peaks to be observed at respective different wavelengths in the visible spectrum when the composition is illuminated under a wavelength of 365 nm, an intensity ratio between a peak of greatest intensity and a peak of smallest intensity being not greater than 20.

6. A composition according to claim 5, in which the intensity ratio between the peak of greatest intensity and the peak of smallest intensity is not greater than 10.

7. A composition according to claim 5, in which the intensity ratio between the peak of greatest intensity and the peak of smallest intensity is not greater than 5.

8. A composition according to claim 5, in which the intensity ratio between the peak of greatest intensity and the peak of smallest intensity is not greater than 3.

9. A composition according to claim 1, wherein a spectral distance between at least two of the three peaks is not less than about 40 nm.

10. A composition according to claim 1, wherein a spectral distance between any two of the three peaks is not less than about 40 nm.

11. A composition according to claim 1, wherein a spectral distance between at least two of the three peaks is not less than about 60 nm.

12. A composition according to claim 1, wherein a spectral distance between any two of the three peaks is not less than about 60 nm.

13. A composition according to claim 1, wherein at least one photoluminescent agent is inorganic.

14. A composition according to claim 1, wherein an intensity ratio between a peak of greatest intensity and a peak of smallest intensity is not greater than 20.

15. A composition according to claim 14, wherein the intensity ratio is not greater than 10.

16. A composition according to claim 14, wherein the intensity ratio is not greater than 5.

17. A composition according to claim 14, wherein the intensity ratio is not greater than 3.

18. A composition according to claim 1, wherein an intensity ratio between any two of the three peaks is not greater than 20.

19. A composition according to claim 18, wherein the intensity ratio is not greater than 10.

20. A composition according to claim 18, wherein the intensity ratio is not greater than 5.

21. A composition according to claim 18, wherein the intensity ratio is not greater than 3.

22. A composition according to claim 1, wherein at least one photoluminescent agent comprises a pigment.

23. A composition according to claim 1, wherein at least one photoluminescent agent comprises a colorant.

24. A composition according to claim 1, wherein at least one photoluminescent agent comprises a metallic salt doped by at least one dopant.

25. A composition according to claim 24, wherein the metallic salt comprises zinc sulfide.

26. A composition according to claim 24, wherein the dopant comprises at least one metal.

27. A composition according to claim 24, wherein the metallic salt comprises particles of mean size smaller than 15 μm.

28. A composition according to claim 27, wherein the mean size is smaller than 10 μm.

29. A composition according to claim 28, wherein the mean size is smaller than 5 μm.

30. A composition according to claim 1, wherein at least one photoluminescent agent comprises at least one of an aluminate or a phosphate doped by at least one dopant.

31. A composition according to claim 30, wherein the dopant comprises at least one rare earth element.

32. A composition according to claim 1, wherein at least one photoluminescent agent comprises at least one rare earth oxide.

33. A composition according to claim 1, wherein at least one photoluminescent agent is particulate, and wherein a mean size of particles of the photoluminescent agent is not greater than about 100 rim.

34. A composition according to claim 33, wherein the mean size is not greater than about 50 nm.

35. A composition according to claim 34, wherein the mean size lies in a range of about 1 nm to about 10 nm.

36. A composition according to claim 1, wherein at least one photoluminescent agent comprises particles coated at least in part in a matrix.

37. A composition according to claim 36, wherein the particles are totally encapsulated in the matrix.

38. A composition according to claim 36, wherein the matrix is polymeric.

39. A composition according to claim 36, wherein the matrix is inorganic.

40. A composition according to claim 36, wherein the matrix includes a refractive index not greater than about 1.8.

41. A composition according to claim 36, wherein the matrix includes a refractive index not greater than about 1.6.

42. A composition according to claim 38, wherein the matrix comprises at least one of polystyrene, polymethylmethacrylate, polyester, polyethylene, or polyether.

43. A composition according to claim 38, wherein the matrix is in particulate form.

44. A composition according to claim 39, wherein the matrix comprises silica.

45. A composition according to claim 39, wherein the matrix comprises silica particles.

46. A composition according to claim 36, wherein the matrix contains particles containing at least two different photoluminescent agents.

47. A composition according to claim 1, wherein a proportion of photoluminescent agents in the composition lies in a range of about 1% to about 50% by weight relative to a total weight of the composition.

48. A composition according to claim 1, wherein a proportion of photoluminescent agents lies in a range of about 5% to about 20% by weight relative to a total weight of the composition.

49. A composition according to claim 1, wherein at least one photoluminescent agent comprises particles having a mean size lying in a range of about 1 nm to about 10 nm.

50. A composition according to claim 1, wherein at least one photoluminescent agent comprises crystals of at least one of CdSe, CdSe/ZnS, or CdTe/CdS.

51. A composition according to claim 1, wherein at least one photoluminescent agent comprises at least one semiconductor.

52. A composition according to claim 51, wherein the semiconductor is selected from the group consisting of Si, ZnO, and CdSe.

53. A composition according to claim 1, wherein the at least one photoluminescent agent causes resulting light emitted by the photoluminescent agents as a result of the illumination to be substantially white light.

54. A composition according to claim 1, wherein the at least one photoluminescent agent comprises more than three photoluminescent agents.

55. A composition according to claim 1, wherein a peak has a wavelength lying in a range of about 620 nm to about 700 nm.

56. A composition according to claim 1, wherein a peak has a wavelength lying in a range of about 592 nm to about 620 nm.

57. A composition according to claim 1, wherein a peak has a wavelength lying in a range of about 578 nm to about 592 nm.

58. A composition according to claim 1, wherein a peak has a wavelength lying in a range of about 500 nm to about 578 nm.

59. A composition according to claim 1, wherein a peak has a wavelength lying in a range of about 446 nm to about 500 nm.

60. A composition according to claim 1, wherein a peak has a wavelength lying in a range of about 400 nm to about 446 nm.

61. A composition according to claim 1, wherein a first peak has a wavelength lying in a range of about 446 nm to about 500 nm, a second peak has a wavelength lying in a range of about 500 nm to about 578 nm, and a third peak has a wavelength lying in a range of about 620 nm to about 700 nm.

62. A composition according to claim 1, wherein a first peak has a wavelength lying in a range of about 400 nm to about 446 nm, a second peak has a wavelength lying in a range of about 500 nm to about 578 nm, and a third peak has a wavelength lying in a range of about 592 nm to about 620 nm.

63. A composition according to claim 1, wherein a first peak has a wavelength lying in a range of about 446 nm to about 500 nm, a second peak has a wavelength lying in a range of about 578 nm to about 592 nm, and a third peak has a wavelength lying in a range of about 620 nm to about 700 nm.

64. A composition according to claim 1, including at least one additional colorant, in addition to the photoluminescent agent.

65. A composition according to claim 64, wherein the additional colorant is selected from the group consisting of a liposoluble colorant, a hydrosoluble colorant, a pigment, and a pearlescent nacre, and mixtures thereof.

66. A packaging device containing a composition as defined in claim 1.

67. A method of applying at least one of a non-therapeutic care product and makeup to at least one of skin, lips, and keratinous fibers, the method comprising applying a composition as defined in claim 1 to at least one of the skin, the lips, and keratinous fibers.
